(19) United States
(12) Patent Application Publication
(10) Pub. No.: US 2014/0349347 A1
(43) Pub. Date: Nov. 27, 2014

(54) SYNTHESIS OF N-ACETYL-D-NEURAMINIC ACID

(71) Applicants: Andreas Schroven, Barssel (DE); Gyula Dekany, Sinnamon Park (AU); Ionnis Vrasidas, Thessaloniki (GR)

(72) Inventors: Andreas Schroven, Barssel (DE); Gyula Dekany, Sinnamon Park (AU); Ionnis Vrasidas, Thessaloniki (GR)

(73) Assignee: Glycom A/S, Kgs. Lyngby (DK)

(21) Appl. No.: 14/364,269

(22) PCT Filed: Oct. 10, 2012
(86) PCT No.: PCT/IB2012/055480

§ 371 Date: Jun. 10, 2014

(30) Foreign Application Priority Data
Dec. 15, 2011 (GB) 1121615.7

(51) Int. Cl. C12P 19/26; C07H 15/22; C07H 15/12

(52) U.S. Cl. CPC C12P 19/26; C07H 15/12; C07H 15/22
USPC 435/84

(57) ABSTRACT

Method of making NANA from NAM, which may itself be made from fructose. The NAM is treated with pyruvic acid or a pyruvate in the presence of a NANA aldolase having at least 70% sequence similarity or homology to the amino acid sequence of SEQ. ID. NO. 1.

### FIELD OF THE INVENTION

The present invention relates to a method for synthesizing N-acetyl-D-neuraminic acid (NANA) from a low cost sugar, such as D-fructose, especially in large amounts.

### BACKGROUND OF THE INVENTION

NANA is a sialic acid which occurs naturally in milk by itself and as a component of oligosaccharides. NANA can be used for making nutritional supplements, particularly for human infant milk formulations, and is also a useful starting material for making compounds of interest as therapeutic agents. However, NANA is currently available only in limited quantities and there has been a need for low cost, efficient processes for its preparation from simple, widely available starting materials.

In bacterial systems, NANA is biosynthesized by a NANA aldolase from N-acetyl-D-mannosamine (NAM) and pyruvate, or by a NANA synthase from NAM and phosphoenolpyruvate. Such enzymes have also been used for chemo-enzymatic syntheses of NANA from pyruvic acid and NAM, synthesized from N-acetyl-D-glucosamine (NAG) via basic epimerization. The epimerization has been carried out with NAG 2-epimerase. See WO 94/29476, EP-A-0428947 and EP-A-578825. NANA has also been chemically synthesized from D-glucose, using oxaloacetic acid. See WO 92/16541.

In developing a commercial process for making NANA, many hurdles have had to be overcome. When NAG has been initially epimerized to NAM with a base, an equilibrium between NAG and NAM has been formed wherein formation of the gluco compound has been favoured. Hence, sophisticated and complicated separation techniques have been needed in such methods to isolate the minor amounts of NAM from the major amounts of remaining NAG starting material. Subsequent in situ use of an aldolase for converting the NAM to NANA has not been beneficial, because the highly alkaline pH (pH>10.5) used for the initial epimerization has been far higher than the optimum pH range needed for the enzyme. Combined use of the epimerase and aldolase has avoided these problems, but the conversion from NAG to NANA and the isolation of NANA has required lengthy and cumbersome isolation/separation procedures and the yields have not been high.

In enzymatic syntheses of NANA from essentially pure NAM, higher yields have been achieved. However, a large excess (appr. 5-10 equiv.) of pyruvate has typically had to be used to push the equilibrium towards the formation of NANA. The remaining unreacted pyruvate has caused isolation problems and decreased the yields of isolated NANA to about 50-60%. The use of even larger excesses of pyruvate has not been an option since such excesses would inhibit the aldolase activity. For this reason, Hu et al. (Appl. Microbiol. Biotechnol. 85, 1383 (2010)) have used an initial pyruvate concentration of 0.5 M and then continuously added more pyruvate when the pyruvate/NANA ratio went below 1.5 (no information given about the total amount of pyruvate used). The isolated yield of NANA was about 70% (no information given about purity).

There has been a need for a better way of industrially producing NANA in high or even higher yields without the need to continuously monitor the production of NANA and continuously adjust the concentration of reactants.

### SUMMARY OF THE INVENTION

The present invention provides a process that can be used for converting fructose into NANA and that can be readily carried out on a large scale for efficient commercial production.

A first aspect of this invention relates to a method for making NANA from NAM, which comprises a step of treating NAM with pyruvic acid or a pyruvate in the presence of a NANA aldolase having at least 70% sequence similarity or homology to the amino acid sequence of SEQ. ID. NO. 1.

Preferably, the NAM used is essentially pure NAM.
Preferably, the NAM is treated with a pyruvate.
A second aspect of this invention relates to a method of making NANA from D-fructose, comprising the steps of:
i) making, from fructose and R₁ NH₂ via a Heyns-rearrangement, an N-substituted D-mannosamine derivative of formula 1

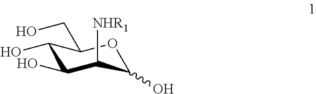

wherein R₁ is a group removable by hydrogenolysis;
ii) hydrogenating and then acetylating the N-substituted D-mannosamine derivative of formula 1 to make NAM; and then
iii) treating the NAM with pyruvic acid or a pyruvate in the presence of a NANA aldolase, preferably a NANA aldolase having at least 70% sequence similarity or homology to the amino acid sequence of SEQ. ID. NO. 1.
Preferably, R₁ is a benzyl or naphthylmethyl group, which may optionally be substituted with one or more phenyl, alkyl or halogen groups. More preferably, R₁ is a benzyl group.
Preferably, NAM is treated with a pyruvate.
Preferably, the NANA aldolase has at least 90% sequence similarity or homology to the amino acid sequence of SEQ. ID. No. 1.

### BRIEF DESCRIPTION OF THE FIGURE

FIG. 1 shows SEQ. ID. NO. 1, which is the sequence of a NANA aldolase of 297 amino acids. This amino acid sequence can be obtained as N-acetylneuraminate lyase EC=4.1.3.3 from E. coli (strain K12) (Kawakami et al. Agric. Biol. Chem. 50, 2155 [1986]).

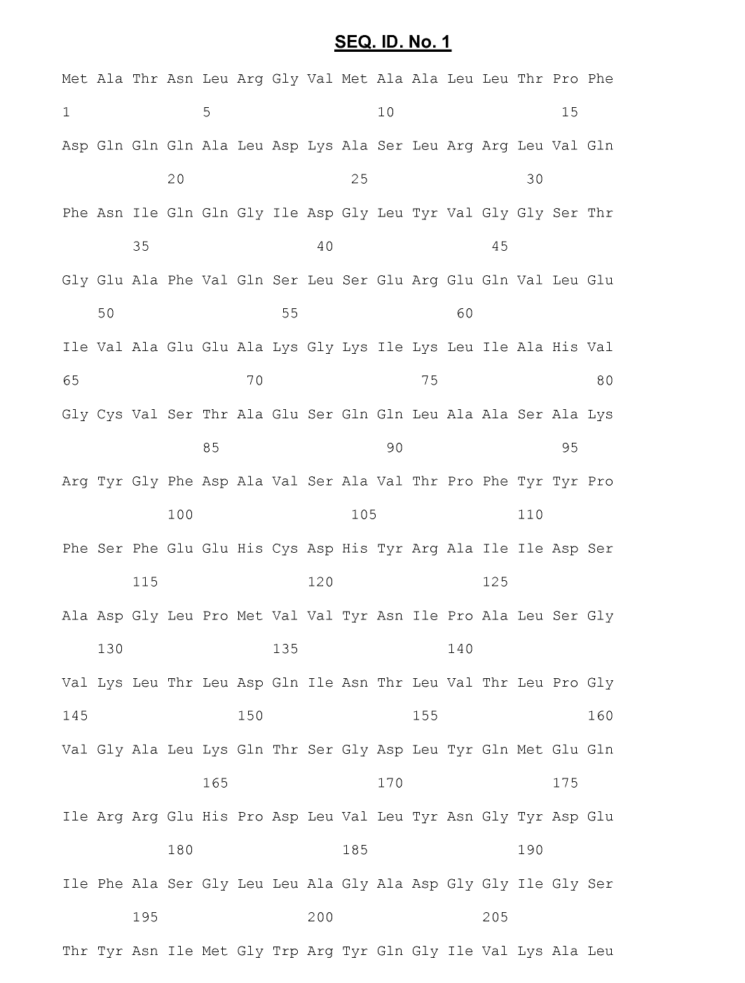

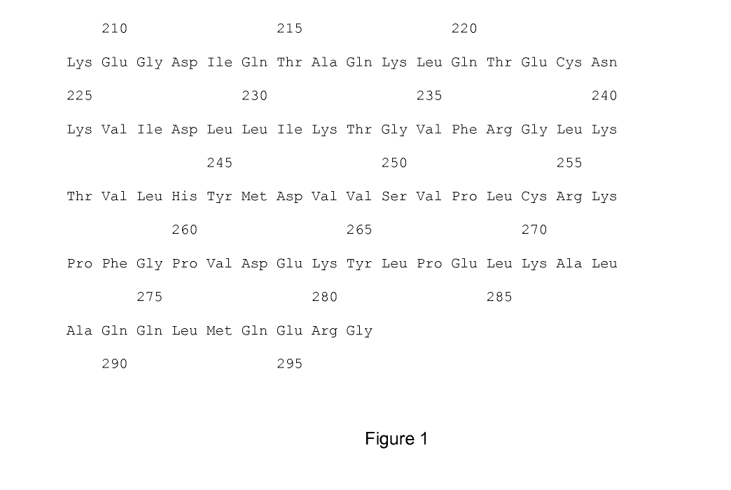

### DETAILED DESCRIPTION OF THE INVENTION

In the present invention, the terminology “group removable by hydrogenolysis” refers to groups whose bond attached to a core carbohydrate structure can be cleaved by addition of hydrogen in the presence of catalytic amounts of palladium, Raney nickel or another appropriate metal catalyst known for use in hydrogenolysis, resulting in the regeneration of the protected functional group, mainly —OH or —NH₂ of the parent molecule. Such protecting groups are well known to the skilled man and are thoroughly discussed in P. G. M. Wuts and T. W. Greene: Protective Groups in Organic Synthesis, John Wiley & Sons (2007). Suitable protecting groups include, but are not limited to, benzyl, diphenylmethyl (benzhydryl), 1-naphthylmethyl, 2-naphthylmethyl or triphenylmethyl (trityl) groups, each of which can optionally be substituted by one or more groups selected from: alkyl, alkoxy, phenyl, amino, acylamino, alkylamino, dialkylamino, nitro, carboxyl, alkoxycarbonyl, carbamoyl, N-alkylcarbamoyl, N,N-dialkylcarbamoyl, azido, halogenalkyl or halogen. Preferably, such substituents, if present, are on the aromatic ring(s). Preferably, these protecting groups are substituted or unsubstituted benzyl groups.

In connection with possible substituents that are borne by a “group removable by hydrogenolysis” as defined above and/or by some of their substituents, the term “alkyl” means a linear or branched chain saturated hydrocarbon group with 1-6 carbon atoms, such as methyl, ethyl, n-propyl, i-propyl, n-butyl, i-butyl, s-butyl, t-butyl or n-hexyl; the term “aryl” refers to a homoaromatic group such as phenyl or naphthyl; the term “acyl” represents an R′—C(═O)-group, wherein R′ may be H, alkyl (see above) or aryl (see above), such as formyl, acetyl, propionyl, butyryl, pivaloyl or benzoyl, and wherein the alkyl or aryl residue may either be unsubstituted or may be substituted with one or more groups selected from alkyl (only for aryl residues), halogen, nitro, aryl, alkoxy, amino, alkylamino, dialkylamino, carboxyl, alkoxycarbonyl, carbamoyl, N-alkylcarbamoyl, N,N-dialkylcarbamoyl, azido, haloalkyl or hydroxyalkyl, giving rise to acyl groups such as chloroacetyl, trichloroacetyl, 4-chlorobenzoyl, 4-nitrobenzoyl, 4-phenylbenzoyl, 4-benzamidobenzoyl, 4-(phenylcarbamoyl)-benzoyl, glycolyl or acetoacetyl; the term “alkyloxy” or “alkoxy” means an alkyl group (see above) attached to the parent molecular moiety through an oxygen atom, such as methoxy, ethoxy or t-butoxy; “halogen” means fluoro, chloro, bromo or iodo; “amino” refers to a —NH₂ group; “alkylamino” means an alkyl group (see above) attached to the parent molecular moiety through an —NH-group, such as methylamino or ethylamino; “dialkylamino” means two alkyl groups (see above), either identical or different ones, attached to the parent molecular moiety through a nitrogen atom, such as dimethylamino or diethylamino; “acylamino” refers to an acyl group (see above) attached to the parent molecular moiety through an —NH-group, such as acetylamino (acetamido) or benzoylamino (benzamido); “carboxyl” denotes an —COOH group; “alkyloxycarbonyl” means an alkyloxy group (see above) attached to the parent molecular moiety through a —C(═O)-group, such as methoxycarbonyl or t-butoxycarbonyl; “carbamoyl” is an H₂N—C(═O)-group; “N-alkylcarbamoyl” means an alkyl group (see above) attached to the parent molecular moiety through a —HN-C(═O)-group, such as N-methylcarbamoyl; “N,N-dialkylcarbamoyl” means two alkyl groups (see above), either identical or different ones, attached to the parent molecular moiety through a >N—C(═O)-group, such as N,N-methylcarbamoyl.

In this invention, the term “essentially pure NAM” means that NAM contains less than 10 w/w % of impurity, preferably less than 5 w/w % of impurity, more preferably less than 2 w/w % of impurity, even more preferably less than 1 w/w % of impurity, most preferably less than 0.5 w/w % of impurity, in particular less than 0.1 w/w % of impurity. By “impurity” is meant any physical entity that is different from NAM, such as unreacted intermediate(s) remaining from the synthesis of NAM, by-product(s), degradation product(s), inorganic salt(s) and/or other contaminants different from organic solvent(s) and water.

In making NANA in accordance with this invention, NAM, preferably essentially pure NAM, is treated with pyruvic acid or preferably a pyruvate, such as sodium pyruvate, potassium pyruvate or calcium pyruvate, preferably sodium pyruvate, in the presence of a NANA aldolase. The NANA aldolase is preferably a NANA aldolase having at least 70%, more preferably at least 80% sequence similarity or homology to the amino acid sequence of SEQ. ID. NO. 1. It is especially preferred that the NANA aldolase has at least 90%, still more preferably at least 95% sequence similarity or homology to the amino acid sequence of SEQ. ID. NO. 1, such as 99%, and it is particularly preferred that the NANA aldolase has the amino acid sequence of SEQ. ID. NO. 1. It has unexpectedly been found by the present inventors that use of this enzyme permits a higher initial concentration of pyruvate or pyruvic acid to be used than is taught in Hu et al., and that continuous addition of pyruvic acid or pyruvate is not needed throughout the reaction in order to push the equilibrium towards the production of NANA. This represents a significant simplification of the method of Hu et al., and thus a cost and efficiency benefit, in particular when seeking to produce NANA on a large, commercial scale.

The NANA aldolase can be used in free or immobilized form to allow reuse of the enzyme. Immobilisation can be carried out, for example, by containment within a hollow fibre or membrane reactor. The conversion of NAM to NANA can be carried out in aqueous medium at a pH of 6 to 9, preferably 7 to 8, at a temperature of 20 to 60° C., preferably 30 to 40° C. The molar ratio of the pyruvic acid or pyruvate relative to NAM is preferably about 1.5:1 to 2.5:1, more preferably about 1.6:1 to 2.0:1, most preferably about 1.8:1 to 1.9:1.

Also preferably, the total amount of pyruvate or pyruvic acid to be present during the treatment step is added in a single step and no further addition of pyruvate or pyruvic acid takes place during the treatment step.

The NANA can be isolated from the reaction mixture by any conventional physical, chemical or physicochemical method, but preferably by crystallization. In this regard, the NANA concentration in the reaction mixture is preferably increased by centrifugation and filtration to at least about 150 g/litre, so that it can then be crystallized from the reaction mixture, e.g. by addition of from 4 to 8, preferably 6, volumes of acetic acid.

In accordance with this invention, an isolated yield of NANA calculated from the starting NAM of about 80% can be achieved, without continuously adding pyruvic acid or pyruvate to the reaction mixture or monitoring the progress of the reaction and without using high excess amounts of pyruvic acid or pyruvate.

In making NANA from D-fructose in accordance with this invention, the N-substituted D-mannosamine derivative of formula 1 is made by a Heyns-rearrangement, which preferably involves the steps of:
a) treating D-fructose with R₁—NH₂ to yield a fructosyl amine derivative,
b) isolating the fructosyl amine derivative as a crude product by removing the excess of R₁—NH₂, and
c) treating the crude fructosyl amine derivative with an acid to obtain the N-substituted D-mannosamine derivative.

In step a), fructose is allowed to react with excess (3-10 equiv.) of primary amine of formula R₁—NH₂. The amine, if a liquid, can serve as solvent, or a concentrated solution of the amine in an alcohol, dioxane, THF, DMF, or another suitable solvent can be used. Preferably, fructose is added to the amine as solvent at about 0° C. and then the mixture is allowed to warm to room temperature or slowly heated up to 40° C. in order that the starting material is consumed. The reaction is continued until consumption of the starting material is observed by TLC, which is typically observed within 24 h, usually within 18-20 hours.

In step b), the excess of reagent is removed before adding acid to initiate the rearrangement reaction. Apolar solvents not dissolving the intermediate fructosyl amine, mainly lower hydrocarbons such as pentanes, hexanes, heptanes or mixtures thereof such as petroleum ether are suitable to extract the reagent amine. As the fructosyl amine derivative formed in the reaction is poorly soluble in apolar solvents, the organic layer containing the reagent may be easily separated. Preferably, the suspension/emulsion formed after addition of the apolar solvent is frozen at a temperature of between −20 and −25 ° C. and the supernatant organic phase is decanted. The supernatant organic phase is found not to contain any significant quantity of carbohydrate-like compound. The washing procedure may be repeated several times. The product must not be precipitated and/or crystallized but should be used directly in step (c). That is to say, the meaning of “isolating the fructosyl amine as a crude product by removing the excess of R₁—NH₂” is that no purification of the fructosyl amine should be carried out other than the removal of the excess reagent, and any additional solvent used in step (a). In particular, the crude fructosyl amine product must not be crystallised. Thereby, one can obtain the manno-epimer (that is, the N-substituted D-mannosamine derivative) from step (c) when using the crude product of step (b).

In step c), the crude fructosyl amine derivative is dissolved in alcohol, dioxane, THF, DMF or a mixture thereof, preferably in an alcohol, more preferably in methanol, and an acid is added to promote rearrangement of the fructosyl amine derivative. The acid may be used in any amount from a catalytic amount to a large excess. The acid may be chosen from the group of inorganic protic acids such as HCl, HBr, sulfuric acid and phosphoric acid, from the group of organic protic acids such as formic acid, acetic acid, oxalic acid, optionally substituted methanesulphonic acid derivatives, optionally substituted benzenesulphonic acid derivatives or polymer bound sulphonic acids (ion exchange resins), or from the group of Lewis-acids such as AlCl₃, ZnCl₂, CuBr₂ and BF₃-etherate. The reaction typically takes place at room temperature and is completed within several hours, such as up to 8 hours, and preferably within 2-4 hours. Two main products are formed, with the major product being N-substituted-glucosamine and the minor component being N-substituted-mannosamine in a proportion of ca. 6:4 to 8:2. The total yield of the two products can be as high as 75-80% based on fructose. The products may be isolated by means of separation techniques such as chromatography. Preferably, step (c) is carried out by dissolving the crude N-substituted fructosyl amine in methanol followed by addition of glacial acetic acid.

Preferably, the amine reagent is an optionally-substituted benzyl amine or optionally substituted naphthylmethyl amine, more preferably benzyl amine, the excess of which is washed away by petroleum ether in step (b), and the resulting crude fructosyl benzyl or naphthylmethyl amine is taken up in methanol in step (c) and is reacted with glacial acetic acid. Also, preferably, Rᵢ-groups in compounds of formula 1 are benzyl or naphthylmethyl groups optionally substituted with one or more groups selected from phenyl, alkyl or halogen.

Alternatively, a compound of formula 1 can be prepared by the method comprising the steps of:
a) treating D-fructose with R₁—NH₂ and a salt thereof,
b) separating a compound of formula 2

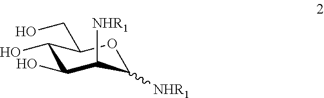

wherein R₁ is a group removable by hydrogenolysis, from the reaction mixture, and
c) converting compound of formula 2 to a compound of formula 1.

In step a), fructose is reacted with an excess (preferably 2-10 equiv., more preferably 3-4 equiv.) of a primary amine of formula R₁—NH₂ in the presence of a salt of the said primary amine. Preferably, the primary amine reagent is an optionally substituted benzyl amine or an optionally substituted naphthylmethyl amine, particularly benzyl amine. The primary amine reagent—if liquid—can also serve as a solvent, or a concentrated solution of the amine reagent in alcohol, dioxane, THF, DMF, or another suitable solvent can be used. The salt of R₁—NH₂ preferably refers to a halide, hydrogen phosphate, N-benzyl-carbamate, bicarbonate or carbonate salt, or carbon dioxide adduct of R₁—NH₂, more preferably benzyl ammonium chloride, and is used in 0.2-1.0 equivalents, preferably 0.3-0.4 equivalents in proportion to the parent amine. Preferably, fructose is added to the amine reagent with the amine reagent acting also as the solvent, followed by the addition of the salt of said amine reagent at about 20-30° C. Alternatively, fructose is added to the mixture of the amine reagent and its salt. The reaction is continued until consumption of the starting material as monitored by TLC, which is typically observed within four days, usually within 48 h, and preferably within 24 hours. At the end of step a), two main products are formed, with the major product being the N,N′-disubstituted 1,2-diamino-1,2-dideoxy-D-glucosamine derivative and the minor component being the N,N′-disubstituted 1,2-diamino-1,2-dideoxy-D-mannosamine derivative in a proportion of ca. 6:4 to 7:3. The total yield of the two products can be as high as 75-100% based on fructose.

In step b) the two isomers are separated by precipitation, crystallization or chromatography. Preferably, in step b) water or aqueous alcohol solution is added to precipitate or crystallize the compound of formula 2 from the reaction mixture, while the gluco-isomer of compound of formula 2 remains in the mother liquor.

In step c) a compound of formula 2 can be easily converted into a compound of formula 1 by treatment with an acid to remove the acid labile —NHR₁ group in the anomeric position and regenerate the anomeric OH. In this reaction water—which is present in the reaction milieu as reagent—may serve as solvent or co-solvent as well. Organic protic or aprotic solvents which are stable under acidic conditions and miscible fully or partially with water such as C₁-C₆ alcohols, acetone, THF, dioxane, ethyl acetate, MeCN, etc. may be used in a mixture with water. The acids used are generally inorganic protic acids selected from but not limited to acetic acid, trifluoroacetic acid, HCl, formic acid, sulphuric acid, perchloric acid, oxalic acid, p-toluenesulfonic acid, benzenesulfonic acid and cation exchange resins, and organic acids including but not limited to acetic acid, formic acid, chloroacetic acid and oxalic acid, which may be present in from catalytic amount to large excess. The hydrolysis may be conducted at temperatures between 20° C. and reflux until reaching completion which takes from about 2 hours to 3 days depending on temperature, concentration and pH. Preferably, the hydrolysis is performed in an alcohol, more preferably in methanol or ethanol, by addition of concentrated HCl or diluted HCl-solution, and the pH is kept at around 3-4. Under such conditions the hydrolysis is typically complete within 2-3 hours at room temperature.

An N-substituted D-mannosamine derivative of formula 1 can be catalytically hydrogenated to form D-mannosamine and then acetylated to form N-acetyl-D-mannosamine (ManNAc) (Scheme 1).

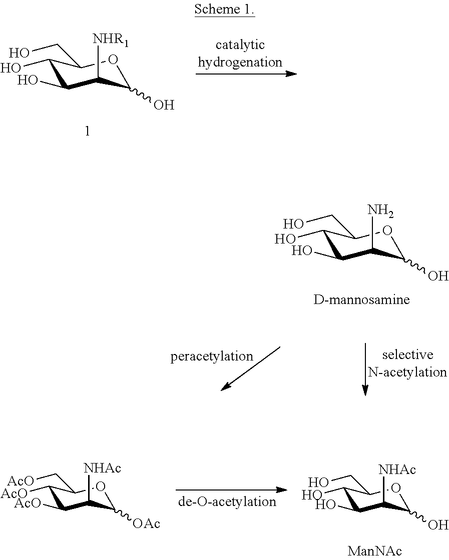

The hydrogenation of the N-substituted D-mannosamine derivative of formula 1 can be carried out in a protic solvent or in a mixture of protic solvents. The protic solvent can be water, acetic acid or a C₁-C₆ alcohol. A mixture of one or more protic solvents with one or more suitable aprotic organic solvents partially or fully miscible with the protic solvent(s), such as THF, dioxane, ethyl acetate or acetone, can also be used. Water, one or more C₁-0₆ alcohols, or a mixture of water and one or more C₁-C₆ alcohols are preferably used as the solvent system. Solutions containing the carbohydrate derivatives in any concentration or suspensions of the carbohydrate derivatives in the solvent(s) used are also applicable. The reaction mixture is stirred at a temperature in the range of 10-100° C., preferably between 20-50° C., in a hydrogen atmosphere of 1-50 bar absolute (100 to 5000 kPa) in the presence of a catalyst such as palladium, Raney nickel or any other appropriate metal catalyst, preferably palladium on charcoal or palladium black, until the reaction is complete. Transfer hydrogenation may also be performed, when the hydrogen is generated in situ from cyclohexene, cyclohexadiene, formic acid or ammonium formate. Addition of organic or inorganic bases or acids and/or basic and/or acidic ion exchange resins can also be used to improve the kinetics of the hydrogenolysis.

As shown in Scheme 1, the acetylation of the resulting D-mannosamine can be carried out in two ways.

The first route is by selective N-acetylation in the presence of one or more hydroxyls. The D-mannosamine can be treated with acetic anhydride, acetyl halogenide or another appropriate acetyl transfer reagent such as acetic anhydride or acetyl chloride. Solvents such as acetone, water, dioxane, DMSO, THF, DMF, alcohols, MeCN and mixtures thereof can be used. The reaction can be carried out in the presence or absence of a base, such as an inorganic base (e.g., K₂CO₃, Na₂CO₃ or NaHCO₃) or an organic base (e.g., pyridine, triethylamine or Hünig's base). The temperature of the reaction can be from −10° C. up to the reflux temperature of the solvent(s). Any overacetylated by-product(s) can be readily transformed into ManNAc with, for example, NaOH/MeOH or NaOMe/MeOH treatment.

The second route is by peracetylation of the D-mannosamine followed by de-O-acetylation. The peracetylation step can be carried out in solution with an acylating agent in the presence or absence of a base. Solvents such as acetone, dioxane, DMSO, THF, DMF, alcohols, MeCN and mixtures thereof can be used. A suitable base such as an inorganic base (e.g., K₂CO₃, Na₂CO₃, NaHCO₃ or NaOH) or an organic base (e.g., pyridine, triethylamine or Hünig's base) can be used. A suitable acylating agent is an activated acetic acid derivative such as acetic anhydride or acetyl chloride. The temperature can be from −10° C. up to the reflux temperature of the solvent(s). The de-O-acetylation step can be carried out in solution in the presence of a base. If the base is an inorganic strong base (e.g., K₂CO₃, LiOH, NaOH, KOH or Ba(OH)₂), preferred solvents are water, alcohol or a mixture of water and an organic solvent (e.g., acetone, dioxane, DMSO, THF, DMF, alcohols or MeCN), and, if the base is an alcoholate (e.g., NaOMe, NaOEt or KO^{t}Bu), the solvent should be the corresponding alcohol (e.g. NaOMe/MeOH). The temperature can be from 0° C. up to the reflux temperature of the solvent(s).

The reduction-acetylation sequence of Scheme 1 can be carried out in separate steps with the isolation of the intermediate D-mannosamine in crystalline form, or in one pot with the acetylation of the crude debenzylated D-mannosamine. Both methods provide pure crystalline NAM in high yield.

The NAM, made from fructose, is then treated with pyruvic acid or preferably a pyruvate, in the presence of a NANA aldolase, preferably as described above. The NANA can then be isolated, preferably by crystallization as described above.

The second aspect of the invention provides a more efficient route from fructose to NANA than those described in the prior art. It is stated by a number of authors that the use of NAM in the enzymatic synthesis of NANA is too expensive to be practical for large scale synthesis (see for example Hu et al. and EP0578825). However, the use of the method of steps (i) and (ii) of the second aspect of the invention to obtain NAM provides a cost effective and efficient route to NAM suitable for large scale synthesis, and, when combined with step (iii), provides a cost effective and efficient route to NANA from readily-available fructose. This avoids the inefficient step of epimerisation of NAG that is required in prior art routes such as those described in EP0578825 and WO94/29476.

Other features of the invention will become apparent in the course of the following examples which are given for illustration of the invention and are not to be limiting thereof.

### EXAMPLES

### 1. N,N′-Dibenzyl-1,2-diamino-1,2-dideoxy-D-mannopyranose

To a mixture of D-(-)-fructose (50 g, 277.5 mmol) in benzyl amine (100 ml) was added benzyl ammonium chloride (30 g, 208 mmol) at room temperature. The mixture was stirred for 6 hours, at which time ethanol (50 ml) was added to make the mixture homogenous. After stirring overnight, another portion of ethanol (50 ml) was added resulting in a homogenous mixture. After 2 hours, water (150 ml) was added and the stirring was continued for 3 hours. The resulting suspension was separated by filtration, and the filtered material was washed with cold aqueous ethanol and dried under vacuum to constant weight, giving 21.3 g of title compound as a white solid. The mother liquor contained N,N′-dibenzyl-1,2-diamino-1,2-dideoxy-D-mannopyranose along with N,N′-dibenzyl-1,2-diamino-1,2-dideoxy-D-glucopyranose.

M.p.: 105-111° C., purity: >95% (by HPLC).

¹H NMR (600 MHz, DMSO) δ: (mixture of α- and β-anomers) H-1 3.90 and 4.40, C₁—NH 2.60, 2.98 and 3.11, C₁—NH—CH₂ 3.68, 3.77, 3.79 and 4.00, Ph 7.16-7.40, H-2 2.77 and 2.82, C₂—NH 1.80, 2.00 and 2.09, C₂—NH—CH₂ 3.74, 3.88 and 4.08, Ph 7.16-7.40, H-3 3.43 and 3.67, C₃—OH 4.66 and 4.74, H-4 3.27 and 3.30, C₄—OH 4.66 and 4.71, H-5 2.94 and 3.45, H-6 3.48, 3.49, 3.58 and 3.68, C₆—OH 4.30 and 4.34.

¹³C NMR (125 MHz, DMSO) δ: (mixture of α- and β-anomers) C-1 83.6 and 87.3, C₁—NH—CH₂ 48.1 and 48.2, C-2 60.4 and 61.5, C₂—NH—CH₂50.7 and 53.9, C-3 70.0 and 75.6, C-4 67.7 and 68.0, C-5 71.9 and 78.2, C-6 61.1 and 61.3, Ph 141.9, 141.1, 140.9, 140.8, 128.1, 128.0, 127.9, 127.7, 126.6 and 126.4.

### 2. N-Benzyl-2-amino-2-deoxy-D-mannose

A) 18.0 g (100 mmol) D-(-)-fructose was treated at 0° C. with freshly distilled benzylamine (3-8 equiv.). The reaction mixture was allowed to warm to room temperature and was then heated at 40° C. for 20 h. The TLC (DCM:MeOH:NH₄OH 2:1:1) showed the disappearance of the starting material. The excess of benzylamine was removed by repeated washings with petroleum ether according to the following method: 150-500 ml of petroleum ether was added into the reaction mixture and subsequently cooled to between −25 and −20 ° C. (dry ice-alcohol bath) until the carbohydrate-rich phase was frozen. The organic layer was then decanted and the procedure was repeated 4-5 times. The obtained crude fructosyl amine was diluted with methanol (200-300 ml) and treated with glacial acetic acid (15-20 ml) at room temperature for 2-4 h. TLC (DCM:MeOH:NH₄OH 20:4:0.5) showed consumption of the ketosylamine and the formation of N-benzyl-2-amino-2-deoxy-D-mannose together with N-benzyl-2-amino-2-deoxy-D-glucose in a ratio of 2:8-4:6 as the main products. The solvent was evaporated under reduced pressure and the residue purified by column chromatography (DCM:MeOH:NH₄OH 20:4:0.5) to give N-benzyl-2-amino-2-deoxy-D-mannose as an amorphous solid (4.1-8.6 g). ¹H NMR (600 MHz, DMSO) δ: α-anomer H-1 5.02 dd, C₁—OH 6.21 d, H-2 2.69 dd, NH 1.97 br, CH₂ 3.82, 3.70 d, Ph 7.17-7.40 m, H-3 3.66 m, C₃—OH 4.50 d, H-4 3.32 m, C₄—OH 4.67 d, H-5 3.51 m, H-6x 3.47 m, H-6y 3.62 m, C₆—OH 4.36 t; β-anomer H-1 4.95 dd, C₁—OH 6.15 d, H-2 2.89 t, NH 2.22 br, CH₂ 3.79, 3.68 d, Ph 7.17-7.40 m, H-3 4.10 m, C₃—OH 4.50 br, H-4 3.67 m, C₄—OH 4.76 br, H-5 3.77 m, H-6x 3.33 m, H-6y 3.57 m, C₆—OH 4.35 t.

¹³C NMR (125 MHz, DMSO) δ: α-anomer C-1 91.3, C-2 61.4, CH₂ 51.8, Ph 141.0, 128.1, 127.9, 126.6, C-3 69.6, C-4 67.7, C-5 72.6, C-6 61.4; β-anomer C-1 101.5, C-2 68.0, CH₂ 51.2, Ph 140.7, 128.1, 127.9, 126.6, C-3 69.0, C-4 69.5, C-5 80.3, C-6 63.6.

B) From compound of Example 1: to a suspension of N,N′-dibenzyl-1,2-diamino-1,2-dideoxy-D-mannopyranose (10 g) in methanol (30 ml) conc. HCl-solution (4 ml) was gradually added and the mixture was heated to 40° C. for 2-3 hours. The solvent was carefully evaporated, the residue was taken up in methanol and evaporated again 3-4 times. The resulting solid was suspended in ethanol and heated to reflux, the insoluble material was removed by hot filtration, and the filtrate was evaporated to dryness to yield the hydrochloride salt of N-benzyl-2-amino-2-deoxy-D-mannose.

### 3. D-Mannosamine hydrochloride

The crude mixture of N-benzyl-2-amino-2-deoxy-D-mannose and N-benzyl-2-amino-2-deoxy-D-glucose obtained in the reaction according to Example 2A before chromatography was suspended in methanol (20-100 ml) and the pH was adjusted to approximately 1-2 with HCl (10-40 ml 2M HCl and additional 2-8 ml conc. HCl). 10% Pd on charcoal (0.4-1.6 g) was added and the reaction mixture was stirred at r.t. or at 45° C. under H₂-atmosphere (up to 5 bars) until all the starting material was consumed. The reaction mixture was filtered through Celite® diatomaceous earth and washed with 20-50 ml of MeOH—H₂O 2:1. The solvents were evaporated until approx. 20-40 ml of volume remained. The crystallization of glucosamine hydrochloride was initiated by adding MeOH (40-80 ml) to the aqueous sugar solution and keeping the solution at 0-5° C. for overnight. The crystals were separated by filtration and washed with cold methanol. Methanol from the mother liquor was evaporated and to the remaining aqueous solution iPrOH (20-80 ml) was added. The solution was kept at 0-5° C. overnight, the crystals formed were filtered, washed with cold iPrOH and dried: 1.3-3.9 g of mannosamine hydrochloride was isolated.

### 4. D-Mannosamine hydrochloride

To N-benzyl-2-amino-2-deoxy-D-mannose (12.0 g) was added a suspension of 10% Pd/C (0.5 g) in 20 ml water. The pH of the reaction mixture was adjusted to 4-4.5 with 10% HCl solution. The reaction mixture was stirred for 6 hours at 40° C. under H₂ pressure (2.5 bars). The pH was then adjusted to 3 with 10% HCl solution. The reaction mixture was kept unstirred overnight so that the catalyst could settle down (the catalyst remains in the reactor). The catalyst is filtered off and washed with small portion of MeOH:H₂O (2:1). The MeOH was removed in vacuo and replaced by 15 ml iPrOH. The iPrOH was distilled off and the same procedure was repeated again. To the reaction mixture 30 ml iPrOH was added and the product crystallized overnight at 4° C. The crystals were filtered and washed with 5-8 ml iPrOH. The wet product was dried at room temperature, to give 8.4 g of D-mannosamine hydrochloride.

### 5. D-Mannosamine hydrochloride

To a suspension of 10 g of N,N′-dibenzyl-1,2-diamino-1,2-dideoxy-D-mannopyranose (Example 1) in 30 ml methanol, 4 ml concentrated HCl was gradually added. The reaction mixture was heated to 40° C. and stirred at this temperature until completion. 10% Pd on charcoal was added as a suspension in 1 ml water. The reaction mixture was placed under H₂, pressurized to 2.5 atm (253 kPa) and heated to 40° C. for 3 hours. The catalyst was then filtered off and washed once with 3 ml methanol: water (2:1). The methanol was removed in vacuo and replaced several times with 10-15 ml isopropanol that was distilled off each time until crystals formed. When crystals formed, the solvent distillation was stopped, and the reaction mixture was stirred at 4° C. for at least 5 hours. The crystals of D-mannosamine hydrochloride were filtered and washed with 2 ml isopropanol and then recrystallized in 2 volumes of ethanol, refluxed for 3-5 minutes and filtered hot.

### 6. N-Acetyl-D-mannosamine (NAM)

A suspension of D-mannosamine hydrochloride (10.0 g) in ethanol-water 6:1 mixture (30 ml) was cooled to 0° C., and triethyl amine (1.2 equiv.) was added at the same temperature. Acetic anhydride (1.2 equiv.) was added dropwise while the temperature was kept between 0-5° C. After completion of the addition (20-30 min) the reaction mixture was seeded with N-acetyl-D-mannosamine crystals and kept at 4° C. under stirring overnight. The crystals formed were filtered, washed and dried to give 9.65 g of 98.5-99% pure N-acetyl-D-mannosamine.

### 7. N-Acetyl-D-neuraminic acid (NANA)

Di-potassium hydrogen phosphate trihydrate (650 mg) and potassium hydrogen phosphate (96 mg) were dissolved in water (71.5 ml) to form a potassium phosphate buffer (pH 7.5). In this buffer, 98.5-99% pure NAM (10 g) from example 6 and sodium pyruvate (9.15 g) were dissolved, and heated to 35° C. The NANA aldolase of SEQ.ID. NO. 1 (143 mg) was added, and the reaction was followed by HPLC to determine the endpoint.

The reaction was stopped after 325 min by the addition of acetic acid (3.6 ml). After stirring for 2 h while allowing the reaction mixture to cool back to room temperature, charcoal (450 mg) and Celite® (450 mg) were added and the mixture was stirred for another 30 min. Charcoal/ Celite® were then filtered out and washed with water. The filtrate was concentrated to 30-40 ml, AcOH (90 ml) at 25° C. was added, and crystallization was initiated by the addition of NANA seeding crystals. The crystallization mixture was stirred at this temperature for 2 h and then cooled to 4° C. Stirring at 4° C. continued for another 11 h. The crystals were filtered and washed with acetic acid and acetone. After drying the solids, 11.3 g of N-acetyl-D-neuraminic acid were obtained with a yield of 81% containing approx. 4-6% of acetic acid measured by NMR.

1. A method for making NANA from NAM and that comprises a step of:
treating NAM with pyruvic acid or a pyruvate in the presence of a NANA aldolase characterized by at least 70% sequence similarity or homology to the amino acid sequence of SEQ. ID. NO. 1.

2. The method of claim 1, wherein the NANA aldolase has a sequence similarity or homology of at least 90% to the amino acid sequence of SEQ. ID. NO. 1.

3. The method of claim 2, wherein the NANA aldolase has the amino acid sequence of SEQ. I.D. NO. 1.

4. The method of claim 1, wherein the NAM contains less than 10 w/w % impurity.

5. The method of claim 1, wherein the NAM is treated with a pyruvate.

6. The method of claim 5, wherein the NAM is treated with sodium pyruvate at a pH of 7 to 8.

7. The method of claim 1, wherein the NAM is treated at a temperature of 30 to 40° C. with a molar ratio of pyruvic acid or pyruvate to NAM of 1.5:1 to 2.5:1.

8. A method of making NANA from D-fructose, comprising the steps of:
i) making, from D-fructose and R₁NH₂ via Heyns-rearrangement, an N-substituted D-mannosamine derivative of formula 1 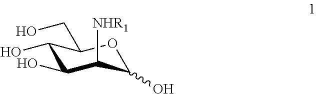
wherein R₁ is a group removable by hydrogenolysis;
ii) hydrogenating and then acetylating the N-substituted D-mannosamine derivative of formula 1 to make NAM; and then
iii) treating the NAM with pyruvic acid or a pyruvate in the presence of a NANA aldolase.

9. The method of claim 8, wherein the NANA aldolase has a sequence similarity or homology of at least 90% to the amino acid sequence of SEQ. ID. NO, 1.

10. The m4ethod of claim 9, wherein the NANA aldolase is characterized by the amino acid sequence of SEQ. I.D. NO. 1,

11. The method of claim 8, wherein the the NAM contains less than 10 w/w % impurity.

12. The method of claim 8, wherein the NAM is treated with a pyruvate.

13. The method of claim 12, wherein the NAM is treated with sodium pyruvate at a pH of 7 to 8.

14. The method of claim 8, wherein the NAM is treated at a temperature of 30 to 40° C. with a molar ratio of pyruvic acid or pyruvate to NAM of 1.5:1 to 2.5:1.

15. The method of claim 8, wherein R₁ is a benzyl or naphthylmethyl group optionally substituted with one or more phenyl, alkyl or halogen groups.

16. The method of claim 15, wherein R₁ is a benzyl group.

17. The method of claim 8, wherein the NANA aldolase has at least 70% sequence similarity or homology to the amino acid sequence of SEQ. ID. NO. 1


SEQUENCE LISTING (1 sequence)

SEQ ID NO: 1 - protein, 297 aa, Escherichia coli
  SOURCE 1..297: /mol_type=“protein” /note=“Strain K-12 N-acetyl neuraminate lyase EC=4.1.3.3” /organism=“Escherichia coli”
MATNLRGVMAALLTPFDQQQALDKASLRRLVQFNIQQGIDGLYVGGSTGEAFVQSLSEREQVLEIVAEEAKGKIKLIAHVGCVSTAESQQLAASAKRYGFDAVSAVTPFYYPFSFEEHCDHYRAIIDSADGLPMVVYNIPALSGVKLTLDQINTLVTLPGVGALKQTSGDLYQMEQIRREHPDLVLYNGYDEIFASGLLAGADGGIGSTYNIMGWRYQGIVKALKEGDIQTAQKLQTECNKVIDLLIKTGVFRGLKTVLHYMDVVSVPLCRKPFGPVDEKYLPELKALAQQLMQERG